(19) United States
(12) Patent Application Publication
(10) Pub. No.: US 2007/0123775 A1
(43) Pub. Date: May 31, 2007

(54) METHOD AND DEVICE FOR MONITORING INFUSIONS

(75) Inventors: Jorg-Uwe MEYER, Ratzeburg (DE); Rainer POLZIUS, Lubeck (DE); Peter TSCHUNCKY, Lubeck (DE); Andreas HENGSTENBERG, Reinfeld (DE); Gotz KULLIK, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: Drager Medical AG & Co. KG, Lubeck (DE)

(21) Appl. No.: 11/533,053

(22) Filed: Sep. 19, 2006

(30) Foreign Application Priority Data
Nov. 25, 2005 (DE) 10 2005 056 310.4

(51) Int. Cl. A61B 5/05

(52) U.S. Cl. 600/427; 600/431; 604/257

(57) ABSTRACT

A method and a device for monitoring infusions at a patient is provided with a dispensing unit (2) for the infusion solution, a labeling substance (3) supplied additionally into the infusion solution by means of a dispensing unit or a drug and with a sensor (5) responding specifically to a physical or chemical property of the labeling substance (3) or of the drug at the body of the patient (1), so that depending on the signal of the sensor (5), an alarm is triggered via an evaluating unit (6) when the signal of the sensor (5) does not detect the presence and/or the quantity of the labeling substance (3) or of the drug, especially after comparison with set values.

### CROSS REFERENCE TO RELATED APPLICATIONS

This application claims the benefit of priority under 35 U.S.C. § 119 of German Patent Application DE 10 2005 056 310.4 filed Nov. 25, 2005, the entire contents of which are incorporated herein by reference.

### FIELD OF THE INVENTION

The present invention pertains to a method and to a device for monitoring infusions at a patient by means of a labeling substance supplied with the infusion solution or with a drug or by means of a change in a physical property, which change is imposed on the infusion solution being supplied.

### BACKGROUND OF THE INVENTION

Only the dispensing is monitored currently in the infusion line outside the patient's body in the medical application of infusion. For example, the infusion pressure and sometimes the volume flow in the infusion line are monitored for this purpose, for example, by means of a drip counter or a thermal volume flow sensor according to German Utility Model G 92 15 265.1 U1. Furthermore, monitoring is embodied in case of stenoses in the infusion line, for example, by checking the overpressure in syringe pumps.

Whether or not the drug does indeed enter the human or animal body and in the calculated quantity despite the fact that it was dispensed is currently not monitored during the infusion of drugs. If, for example, the cannula used for intravenous access is not placed securely, the drug being transported with the infusion solution does not enter the body and the error is not recognized either by monitoring the pressure or the volume flow of the dispensing unit.

### SUMMARY OF THE INVENTION

Thus, the object of the present invention is to provide a reliable method with a device for monitoring the mass flow of the drug being actually infused or of the infusion solution into the patient's body.

According to the invention, a labeling substance or a drug, whose presence and/or quantity is measured in the patient, is supplied by means of the infusion solution, so that the presence and/or the quantity of the infused infusion solution is determined from the quantity of the labeling substance measured in the patient.

According to another aspect of the invention, infusions are monitored at a patient by means of a change in a physical property, which is imposed on the infusion solution being supplied and is measured in the patient.

A physiologically harmless labeling substance or a drug, whose presence and/or quantity in the body is determined by means of a specifically responding sensor, is preferably added to the infusion solution.

Specifically responding sensors for checking the infusion are operated especially in a wireless manner, for example, by radio. The labeling substances may be, for example, nanoparticles or dyes.

A possible change in a physical property of the infusion solution is, for example, a rise in temperature immediately in front of the cannula and a temperature measurement at the vein directly downstream of the cannula in terms of flow. The imposed rise in temperature may also be modulated dynamically, for example, in the form of a cyclic pulse or pulse pattern. Labeling substances may be, for example, optical or magnetic “contrast media,” for example, “ICG-Pulsion” from Pulsion Medical Systems AG with indocyanine green as the active ingredient, or nanocrystalline magnetic particles with a magnetic iron oxide core. Such labeling substances are either extensively metabolized by the body during the circulation or are not metabolized at all and are rapidly excreted in the intact form. Labeling substances can be detected by physical or chemical analytical methods.

If magnetic labeling substances or a drug with inert magnetic nanoparticles are administered with the infusion, these can be detected by means of an especially inductive magnetic detector after entering the blood vessels at a short distance behind the entry site of the infusion. Due to the elimination of labeling substances in the form of, for example, nanoparticles in the blood stream by cellular processes, unambiguous reference to the infusion method is guaranteed, because reflux of the magnetic labeling substances or magnetic nanoparticles to the infusion site is ruled out. Especially preferred are larger nanoparticles with a mean particle size of at least about 300 nm, which are eliminated more rapidly from the body than smaller particles. To increase the rate of elimination, molecular anchors can also be fixed on the surface of the nanoparticles if necessary.

The magnetic detector is arranged in the vicinity of the entry site of the infusion on the skin surface of the patient and makes possible in this way the non-invasive monitoring of the infusion method.

Low-molecular-weight substances, which can be detected by means of absorption or fluorescence detection on account of their optical properties, are especially suitable for use as optical labeling substances. Fluorophores, which can be quenched by binding to constituents of the blood, are preferably used.

An indicator dye, which affects the signal of oximetry, which is known per se, for the infrared optical measurement of the oxygen partial pressure, may be added to the infusion solution by means of an additional dispensing means. For example, indocyanine green, methylene blue and indigo carmine extinguish the signal of the oximeter. If the needle is dislocated, the physiological signal of the venous blood stream appears.

Furthermore, the drug being dispensed is suitable for use as a labeling substance without special, separately added labeling substance if it has a characteristic optical infrared absorption spectrum. The presence of the drug can be measured now by means of a reflection type infrared measuring sensor in the vicinity of the entry site of the infusion.

Another possible labeling substance is dissolved oxygen at a known concentration. The quantity of the infused liquid and the quantity of the infused active ingredient are then calculated from the difference in concentrations in the venous blood stream at a short distance in front of the entry of the infusion into the body and at a short distance thereafter. The method provides for the enrichment of the infusion solution with added oxygen, the increase in oxygen saturation of hemoglobin being measured by means of an oximeter operating according to the reflection principle. The infusion solution may be cyclically enriched with oxygen in order to avoid absolute measured values or a quantitative evaluation and to detect relative changes only.

Due to the specific design of the tip of the infusion needle, for example, with an ultrasound source, an oscillating physical change is induced in the tip of the needle as a consequence of the transportation of liquid. The oscillating signal, for example, a change in pressure, is picked up by a detector, which is fixed on the skin surface in the area in which the tip of the infusion needle is positioned. Quantitative evaluation is also possible, since the oscillating signal is correlated with the volume flow of the infusion/drug solution being infused.

As an alternative, a sound signal is coupled into the infusion system in the area of the dispensing unit for the infusion solution. Since liquids conduct sound well, the sound signal can be detected proximally at the vein by coupling the sound signal into the venous blood stream. A statement can thus be made on the connection between the dispensing unit and the venous blood stream. The sound coupling may take place via a piezo transducer.

As an alternative, an electrode couples for an impedance analysis an electric alternating pulse, which is preferably modulated such that it is coupled with the quantity of the active ingredient being dispensed with the infusion solution. This coupled alternating pulse is recorded by a detector electrode on the patient's body via the electrically conductive infusion solution. The measured signal is used as an indicator for whether infusion is indeed being introduced into the patient's body.

Another alternative of the method is that very fine gas bubbles (microbubbles) are generated in the area of the dispensing unit for the infusion solution and added to the infusion solution in proportion to the quantity of the active ingredient/drug to be dispensed. These microbubbles are detected by an ultrasound measuring sensor, which is arranged proximally to the entry site of the infusion. The marked reflection of the coupled ultrasound signal at the phase boundary of the microbubbles due to the great difference in density is utilized here. As an alternative, ultrasound-specific labeling substances such as “Levovist” based on 99.9% galactose and 91% palmitic acid are used as contrast media.

In case of invasive measurement at the patient's body, a needle electrode is used, in particular, the labeling substance consisting of a physiologically harmless substance, especially one or more of the following: Oxygen, a pH-active substance, a salt or sugar, and aspirin. The labeling substance is dispensed into the infusion solution simultaneously with the drug proper. The substances or the effects produced by them in the patient's blood, for example, a change in the pH value, are measured by means of a needle electrode. The needle electrode may be combined with the infusion cannula in order to get by with one invasive access.

Another method variant without additional labeling substance provides for coupling an electric alternating pulse, which is modulated especially corresponding to the quantity of the infusion solution being dispensed, into the infusion system by means of an electrode. The alternating pulse is recorded via the electrically conductive infusion solution by means of standard ECG (Electrocardiogram) electrodes and evaluated by means of the ECG evaluating unit. The detected signal is used as an indicator showing whether infusion is being carried out and if yes, at what rate of dispensing.

In another method variant without additional labeling substance, the dispensing unit for the infusion solution modulates pressure pulses into the infusion line as a function of the quantity of infusion being dispensed. These pressure pulses are recorded by blood pressure measurement, which is known per se, and are evaluated by the hemodynamic monitoring. The measured signal is used as an indicator showing whether infusion is being carried out and if yes, at what rates.

Another imposed change of a physical property is the pressure, which becomes established in the infusion system and varies depending on whether the cannula is placed correctly or is dislocated. The pressure difference between the venous blood pressure at the cannula and the atmospheric air pressure is evaluated and is used as an indicator showing whether infusion is taking place, but without quantitative evaluation of the quantity of the drug/active ingredient being infused.

It is often especially advantageous in the above-described method that monitoring already present at the patient is used and no special sensory mechanism and/or no special evaluating unit is needed. One example is the use of the existing hemodynamic blood pressure monitoring for monitoring the infusion.

Exemplary embodiments of two devices, shown schematically, for carrying out a method for monitoring infusions at a patient will be explained below on the basis of the two figures.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a method for monitoring infusions at a patient; and

FIG. 2 is a schematic view showing a variant of the method for monitoring infusion at a patient of FIG. 1.

### DESCRIPTION OF THE PREFERRED EMBODIMENTS

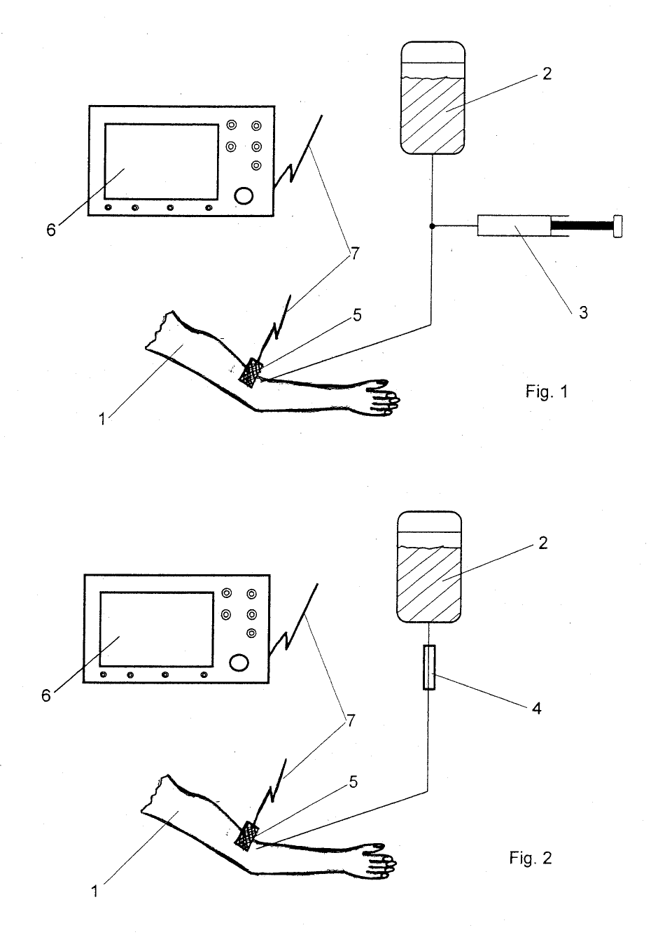

Referring to the drawings in particular, according to FIG. 1, a patient 1 is supplied with an infusion solution intravenously by means of a dispensing unit 2. A labeling substance 3, for example, an optical indicator dye, is dispensed into the line leading to the patient 1 by means of an additional dispensing means. A sensor 5 at the patient 1, which is operated in a wireless manner and is especially capable of triggering an alarm, evaluates the signals in the immediate vicinity downstream of the infusion site. The signal is transmitted from the sensor 5 to the evaluating unit 6 by means of the antennas 7 shown symbolically.

Depending on the signal of the sensor 5, the evaluating unit 6 triggers an alarm when the signal of the sensor 5 fails to detect the presence and/or the quantity of the labeling substance 3 being dispensed. The corresponding desired value of the labeling substance 3 being dispensed is stored in the evaluating unit 6.

While the components are otherwise the same, a signal transmitter 4 is arranged in the infusion line leading to the patient 1 instead of an additional dispensing means for a labeling substance 3 in FIG. 2 for carrying out the method for monitoring infusions in an alternative manner. A specific change in a physical parameter of the infusion solution is imposed on the infusion solution by means of the signal transmitter 4. For example, the specific change in a physical parameter of the infusion solution imposed on the infusion solution may be a pulse pattern with modulation. This is imposed by means of a piezo transducer.

As an alternative, an electric alternating pulse is imposed on the infusion solution by means of electrodes. The corresponding measurement at the patient 1 again takes place by means of a sensor 5 transmitting signals to an evaluating unit 6 in a wireless manner, namely, either by means of a sensitive sound or pressure sensor or an electrode array at the infusion site of the patient.

Depending on the signal of the sensor 5, an alarm is triggered by the evaluating unit 6 when the signal of the sensor 5 does not detect the presence of the imposed variable physical property (any longer) or it remains below a preset desired or reference value.

While specific embodiments of the invention have been shown and described in detail to illustrate the application of the principles of the invention, it will be understood that the invention may be embodied otherwise without departing from such principles.

What is claimed is:

1. A method for monitoring infusions at a patient, the method comprising:
providing a labeling substance;
supplying the labeling substance with an infusion solution or with a drug to the patient; and
measuring the presence and/or quantity of the labeling substance at the patient to determine the presence and/or the quantity of the infused infusion solution or to determine the presence and/or the quantity of the drug measured at the patient.

2. A method in accordance with claim 1, wherein said labeling substance or the drug can be determined by its physical properties.

3. A method in accordance with claim 2, wherein said labeling substance or the drug can be determined by magnetic, electric, acoustic or optical properties.

4. A method in accordance with claim 1, wherein said labeling substance or the drug is determined by using a sensor responding specifically to physical properties of the labeling substance or of the drug.

5. A method in accordance with claim 1, wherein said labeling substance or the drug consists of nanoparticles, especially chemically inert nanoparticles with a mean particle size of at least about 300 nm.

6. A method in accordance with claim 1, wherein said labeling substance consists of physiologically harmless substances that can be determined on the basis of their chemical composition.

7. A method in accordance with claim 1, wherein said labeling substance consists essentially of one or more substance selected from the group comprising oxygen, a sugar, a salt, aspirin and/or a pH-active substance.

8. A method in accordance with claim 1, wherein said labeling substance or the drug being supplied is measured non-invasively at the blood vessel downstream of the entry site of the infusion into said patient.

9. A method for monitoring infusions at a patient, the method comprising:
providing an infusion substance;
imposing a change in a physical property, which change is imposed on the infusion substance;
supplying the infusion substance with the changed physical property to the patient; and
measuring the changed physical property at the patient.

10. A method in accordance with claim 8, wherein the physical property is one or more property of the infusion substance selected from the group temperature, electric properties, electromagnetic properties, acoustic properties or optical properties.

11. A method in accordance with claim 8, wherein the imposed change takes place variably over time in the form of one of a cyclic pulse or pulse pattern with modulation.

12. A method in accordance with claim 8, wherein the imposed change in the physical property of the infusion solution is measured by means of a non-invasive sensor at the body of the patient.

13. A method in accordance with claim 12, wherein the measurement by means of a non-invasive sensor at the body of said patient is at a blood vessel of the patient downstream of the entry site of the infusion into the patient.

14. A method for monitoring infusions of an infusion substance at a patient, the method comprising:
providing the infusion substance with an indicator that is one of a labeling substance provided with an infusion solution or with a drug as the infusion substance, or is a change in a physical property of the infusion solution or of the drug as the infusion substance; and
measuring the presence and/or quantity of the indicator at the patient, so that the presence and/or the quantity of the infused infusion substance is determined.

15. A device for monitoring infusions of an infusion substance at a patient, the device comprising:
a dispensing unit for dispensing the infusion substance;
a labeling substance supplied into the infusion solution or into a drug;
a sensor responding specifically to a property of said labeling substance or of the drug at the body of said patient; and
means for alerting for providing an alarm triggered, depending on the signal of said sensor, when the signal of said sensor does not detect the presence and/or the quantity of said labeling substance or of the drug.

16. A device according to claim 15, wherein said alarm is triggered after a comparison with set values.

17. A device for monitoring infusions of an infusion substance at a patient, the device comprising:
a dispensing unit for dispensing the infusion substance;
means for imposing a change in a physical property, which is imposed on the infusion substance;
a sensor responding to the physical property at the body of the patient; and
means for alerting for providing an alarm triggered, depending on the signal of said sensor, when the signal of said sensor does not detect the presence of the physical property or the degree of presence of the physical property.

18. A device according to claim 17, wherein said alarm is triggered after a comparison with set values.

19. A device according to claim 17, wherein said means for imposing a change in a physical property is a signal transmitter.
